(19) United States
(12) Patent Application Publication
(10) Pub. No.: US 2001/0006794 A1
(43) Pub. Date: Jul. 5, 2001

(54) SCREENING FOR MODULATORS OF BIOMOLECULES

(76) Inventors: AMY BOGGS, MENLO PARK, CA (US); KEITH BOSTIAN, MENLO PARK, CA (US); FRANCOIS MALOUIN, LOS GATOS, CA (US); THOMAS PARR, MONTERA, CA (US); MOLLY SCHMID, SARATOGA, CA (US)

Correspondence Address: LYON & LYON LLP, SUITE 4700, 633 WEST FIFTH STREET, LOS ANGELES, CA, 90071-2066, US

(21) Appl. No.: 09/059,802

(22) Filed: Apr. 14, 1998

Related U.S. Application Data
(63) Continuation of application No. 08/377,329, filed on Jan. 23, 1995, now abandoned.

(51) Int. Cl. C12Q 1/02; G01N 33/53; C12Q 1/04; A61K 49/00

(52) U.S. Cl. 435/29; 435/34; 435/7.7; 424/9.2

(57) ABSTRACT

Method for screening for a modulator of a biomolecule by comparing growth of a first microbe having an altered biomolecule with a second microbe having a normal biomolecule. The first and second microbes are grown in contact with a potential modulator in a growth medium.

### BACKGROUND OF THE INVENTION

This invention relates to methods for screening for enhancers or inhibitors of biomolecules such as enzymes or RNA molecules and the like.

### SUMMARY OF THE INVENTION

This invention features a method for rapid screening of modulators of biomolecules in a sensitive, specific, and highly informative manner. Specifically, the method allows multichannel screening by use of mutated microbes as indicators of useful modulators. Examples of such mutants include temperature-sensitive mutants which are more susceptible to agents that act on the mutated biomolecule. Such biomolecules include enzymes such as DNA gyrase and RNA activators. The method allows rapid screening of several different biomolecules simultaneously and provides an indication of the target(s) of the agent.

Thus, in a first aspect the invention features a method for screening for a modulator of a biomolecule. The method includes comparing the growth of a first microbe having an altered biomolecule with that of a second microbe having a normal biomolecule. Both the first and second microbes are grown in contact with a potential modulator in an appropriate growth medium.

By “screening” is meant that the previously unknown properties of interest of a molecule are determined in the assay. This procedure is distinct from an individual test to determine the properties of such a molecule. Generally, the method includes screening of a large number of potential modulators simultaneously, for example, 5 or 50 or more such modulators. Those in the art will recognize that potential modulators include a wide variety of biochemical molecules including small molecules of molecular weight less than three thousand, as well as larger molecules including oligonucleotides, peptides, lipids, and carbohydrates.

A modulator is an agent which is able to affect the activity of a biomolecule by either inhibiting or enhancing that activity. Generally, such a modulator is an inhibitor of the biomolecule.

By “biomolecule” is meant any molecule that is present within a living organism, including proteins, peptides, polypeptides, carbohydrates, lipids, RNA, DNA, and oligonucleotides. Generally biomolecules utilized in this invention are enzymes, or activators of RNA metabolism.

An altered biomolecule is one that differs from that present in the naturally occurring microbe, i.e., a normal microbe. By “altered” is meant that the biomolecule is either defective in its activity, that is, it has a reduced level of activity (e.g., 20% reduced, but preferably not completely diminished activity), or has an enhanced activity, that is, an activity some fold (e.g., at least 25% more) greater than that found in the normal biomolecule. The term also includes a defect in amount— e.g., overexpression or underexpression of the biomolecule. Examples of such biomolecules include DNA gyrases as exemplified below. Applicant has determined that such altered biomolecules are more sensitive to agents which act at those molecules, for example, a DNA gyrase is more susceptible to fluoroquinolones. Such greater susceptibility allows more sensitive detection of agents, i.e., modulators, which act at those biomolecules. Thus, applicant has determined that potential modulators of a particular enzyme can be readily screened in a rapid assay for activity at such molecules using microbes with defective biomolecules.

By “microbe” is meant to encompass generally haploid organisms such as bacteria, fungi, and viruses, for example, yeast, Escherichia coli, Staphylococcus aureus, and the like. Such organisms, generally having only one form of any gene, are thus more readily manipulated by genetic means.

In the method, the growth of the microbes is compared. This means that a direct or indirect measure of such growth can be used. For example, the turbidity of a medium can be monitored by standard procedures, the pH of the medium monitored, or the viability of cells monitored with a fluorophore. Those in the art will recognize that other direct or indirect methods of measuring growth of the microbes are within the scope of this invention.

In a preferred embodiment, the method further includes comparing the growth of the microbes in a plurality of different media which differ in their carbon source. Applicant has determined that the ability of the microbe having an altered biomolecule, to utilized various carbon sources for growth, is indicative of the mechanism of action of agents or modulators on that microbe. That is, in microbes having altered biomolecules those microbes are under stress and may have a reduced central function. This stress on the microbe alters its ability to use one or more carbon sources in a manner which reflects the mechanism of utilization of those sugars. Such a method allows a fingerprint of the activity of modulators of the biomolecules and allows analysis of the mechanism of action of the modulators based on the carbon utilization of the microbe. Examples of such analyses are provided below.

Thus, in another aspect the invention features a method for determining a fingerprint of a microbe having a biomolecule by comparing the growth of one or more microbes having an altered biomolecule in a plurality of different media differing in their carbon source.

In particular embodiments of the above aspects, the microbe is selected from a group consisting of the Staphylocci, the Pseudomonads, the Enterococci, and the Streptococci. Of particular interest are the common pathogenic species, such as Staphylococcus aureus, Pseudomonas aeruginosa, Enteroccus faecium, Enterococcus faecalis, and Streptococcus pneumoniae.

Also in particular embodiments, the expression level of the altered biomolecule is different from the expression level of the wild-type homolog of that biomolecule in the parent strain of the microbe. The altered biomolecule can be overexpressed, that is, expressed at a significantly (20% or greater) higher level than in the parent microbe strain. Alternatively, in other embodiments, the altered biomolecule can be expressed at a lower level (20% less or lower), that is, less of the altered biomolecule is produced than the wild-type homolog in the respective microbe strains. One approach for obtaining such altered expression level is by the cloning of a regulatory sequence which is transcriptionally-linked with the gene for the altered biomolecule and which results in a change in the level of transcription and/or translation of that gene.

Other features and advantages of the invention will be apparent from the following description of the preferred embodiments thereof, and from the claims.

### DESCRIPTION OF THE PREFERRED EMBODIMENTS

The drawings will first briefly be described.

Drawings

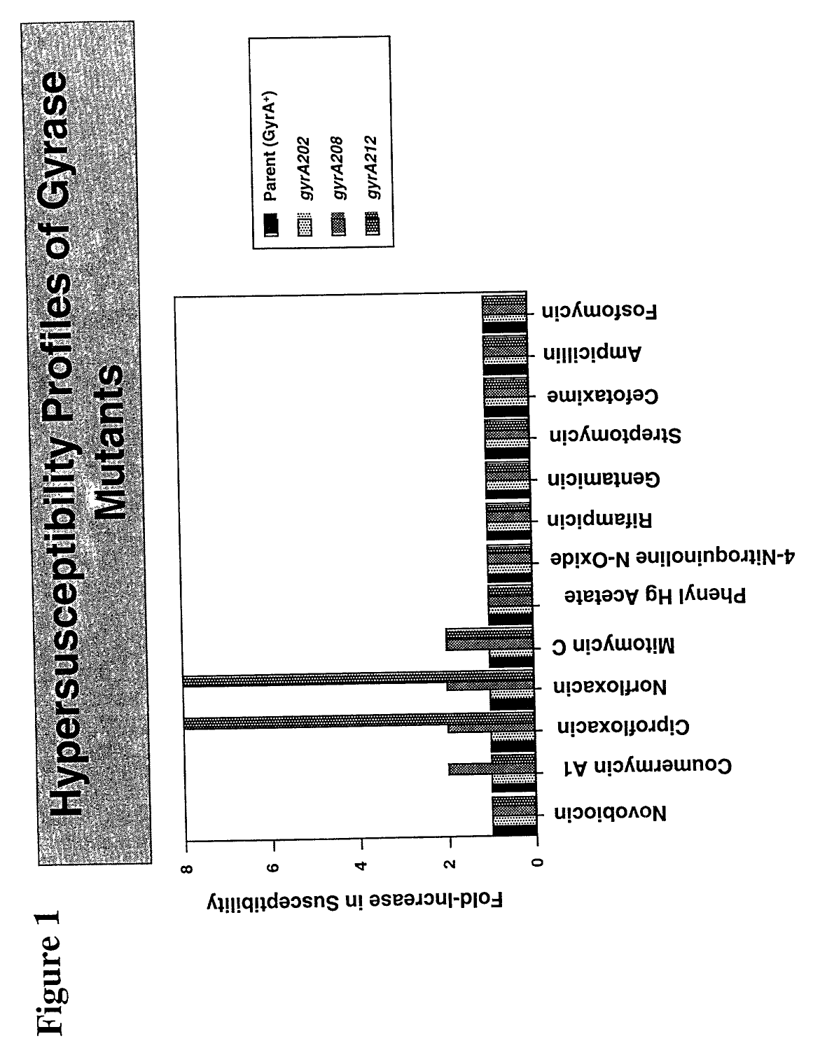

FIG. 1: Hypersusceptibility Profiles of Gyrase Mutants

A Salmonella typhimurium parent strain 7389 was used to form a collection of temperature sensitive mutants. Each mutant exhibited a lethal phenotype at the nonpermissive temperature. Complementation of the mutant phenotype was done. In the case of the three mutants described in the data of this figure (gyrA202, gyrA208, gyrA212) the complemented mutants were identified to be in the A subunit of the bacterial DNA gyrase enzyme. Each mutant was altered in a different portion (see number of mutant) of the A subunit. NB. the A subunit is the protein to which flouroquinolones (e.g. ciprofloxacin, norfloxacin) interact to bring about their antimicrobial activity. FIG. 1 displays data accumulated in the examination of relative susceptibility of the mutants and their parent to various antimicrobial agents. The data are expressed as fold increases in susceptibility. A four-fold increase in susceptibility is the minimum change to be considered significant in this example. These data show a specific increase in susceptibility (8-fold) to only the DNA gyrase inhibitors norfloxacin and ciprofloxacin to mutant gyrA212.

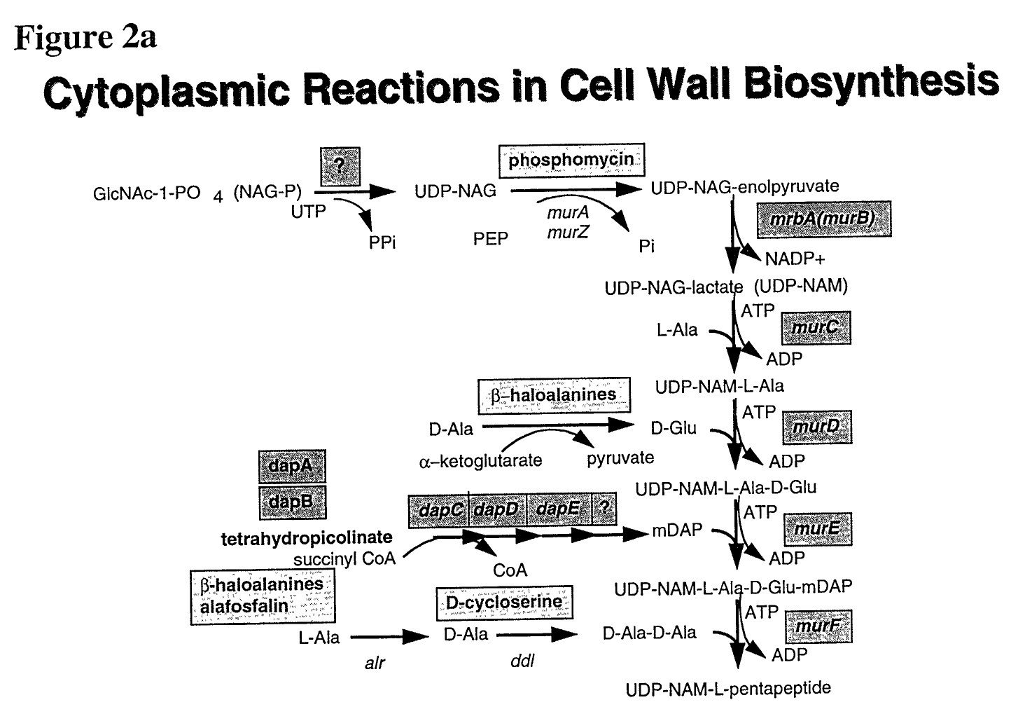

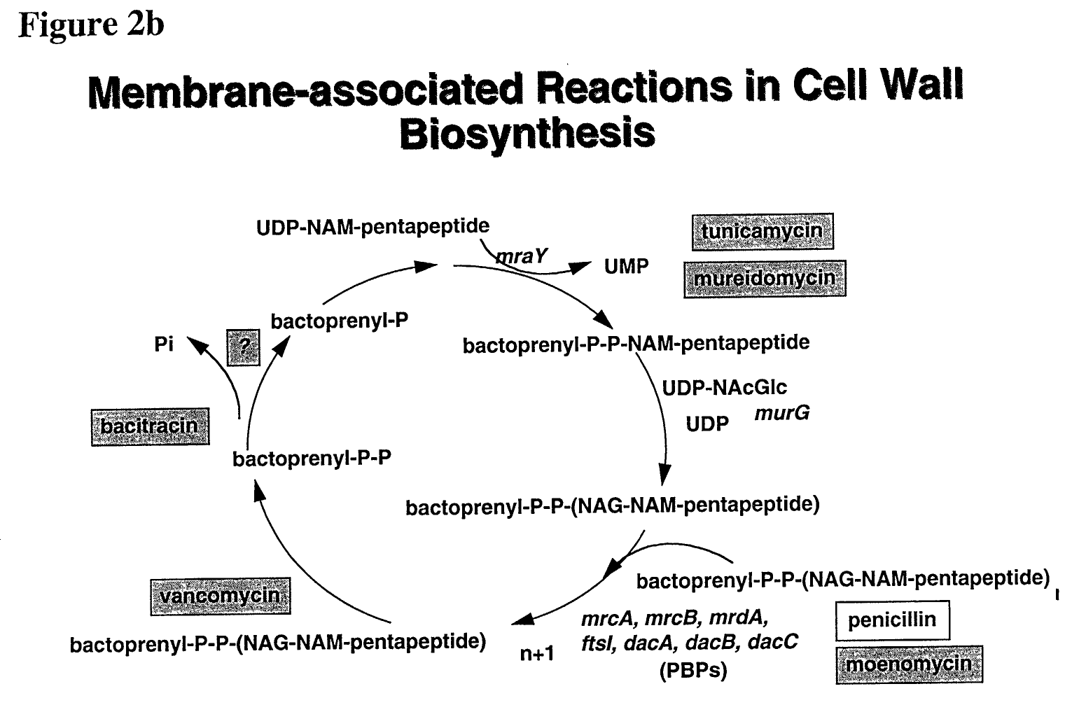

FIG. 2: Cytoplasmic reactions in cell wall biosynthesis.

The process of cell wall biosynthesis has been extensively characterized. Many, but not all of the enzymatic steps in the process have been identified. Specific enzymes are in some case known. The process of synthesis of the intermediate UDP-NAM-L-pentapeptide involves a number of known and unknown genes and proteins. Inhibitions, through for example a temperature sensitive partially functioning enzyme in one step of the pathway will influence other phenotypes in the pathway. As example a partially functioning enzyme such as murD might be expected to influence the susceptibility to antibiotics known to interact either upstream or down stream of the partially functioning enzyme. For example, phosphomycin susceptibility and penicillin susceptibility may be differentially effected by the murD. These two agents act on cell wall biosynthesis either before (phosphomycin) [FIG. 2a] or after (penicillin) [FIG. 2b] the step in cell wall biosynthesis where the partially functioning enzyme is positioned. This differential susceptibility within the biochemical pathway can be observed and this difference can be used as a tool for screening for inhibitors which show this desirable phenotype (inhibition of cell wall biosynthesis). This is an example of a specific indicator mutant which can be incorporated into a multichannel screen.

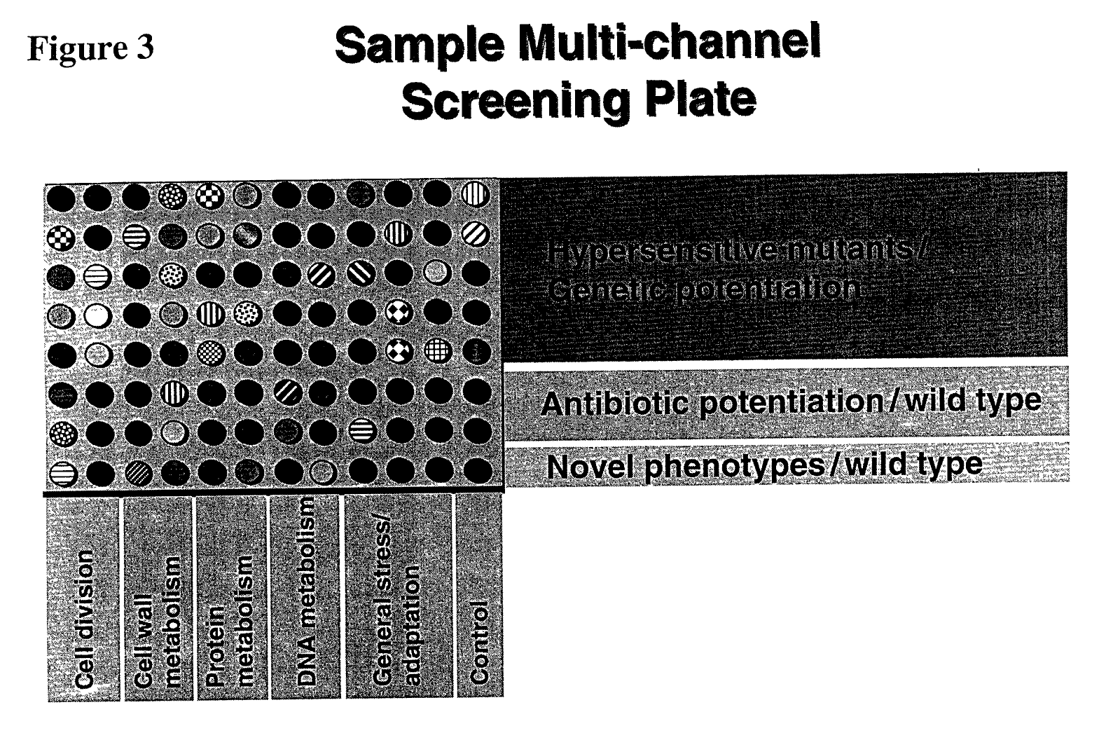

FIG. 3 is a cartoon of a sample multi-channel screening plate. In this example there are depicted three kinds of tests. The first and major portion of the plate is a collection of hypersensitive mutants, as per the DNA gyrase example. Collections of such mutants are grouped based on metabolic associations, so for example one grouping would be including hypersensitive mutants involved in cell wall synthesis, another in protein metabolism and so forth. A second group of tests involves the potentiation of known antibiotics, known antimicrobial agents are combined at subinhibitory concentrations in these wells with mutant cells. These tests exemplify potentiation of wild-type and mutant test cells. Finally there are a collection of novel phenotypes, phenotypes of interest due to their (for example) rapidly bactericidal effects.

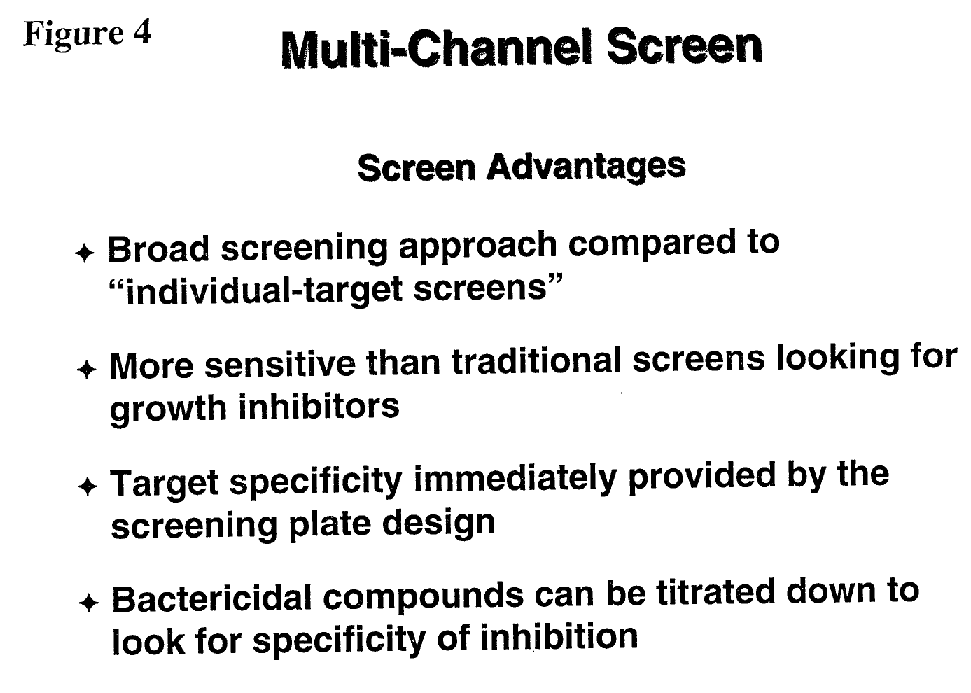

FIG. 4 lists some of the advantages of multi-channel screening. This list is incomplete but provides some examples of distinctive characteristics which are not found in conventional biochemical screening methods.

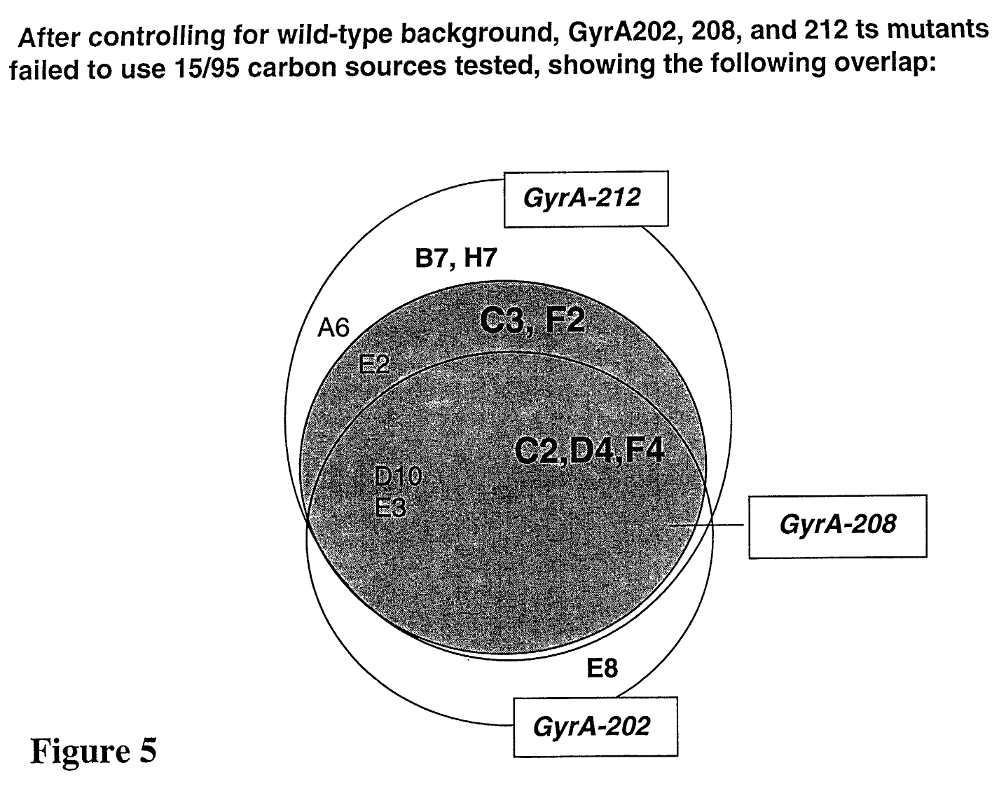

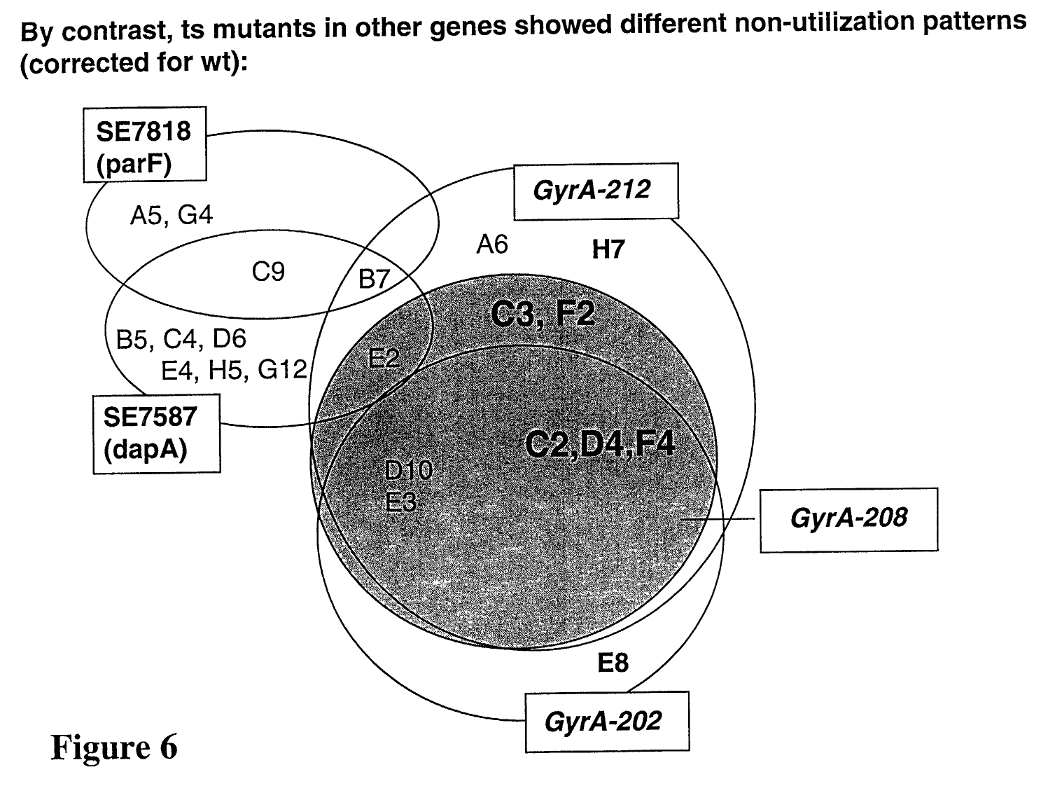

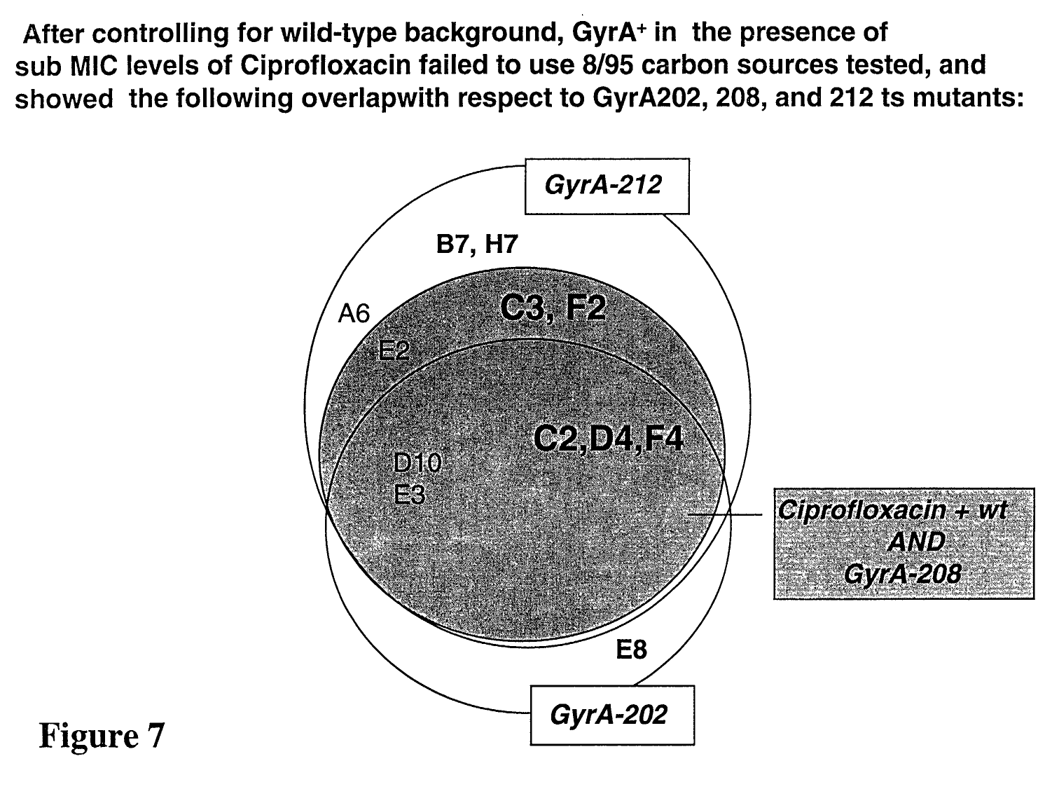

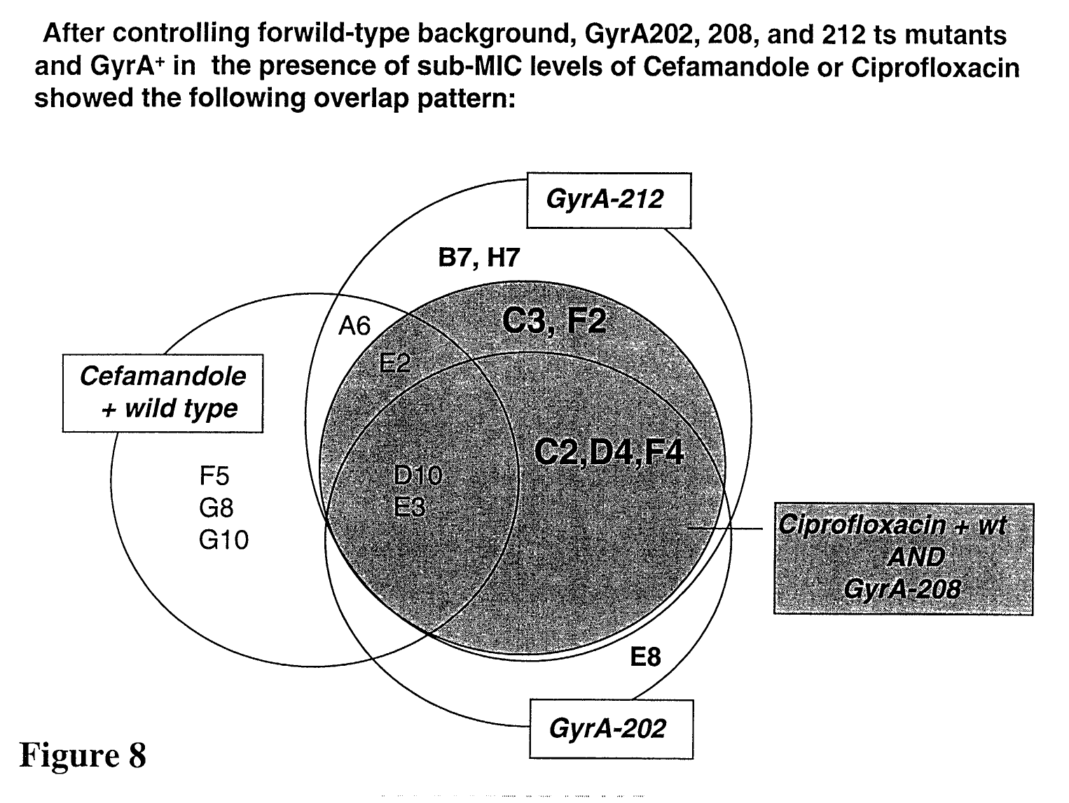

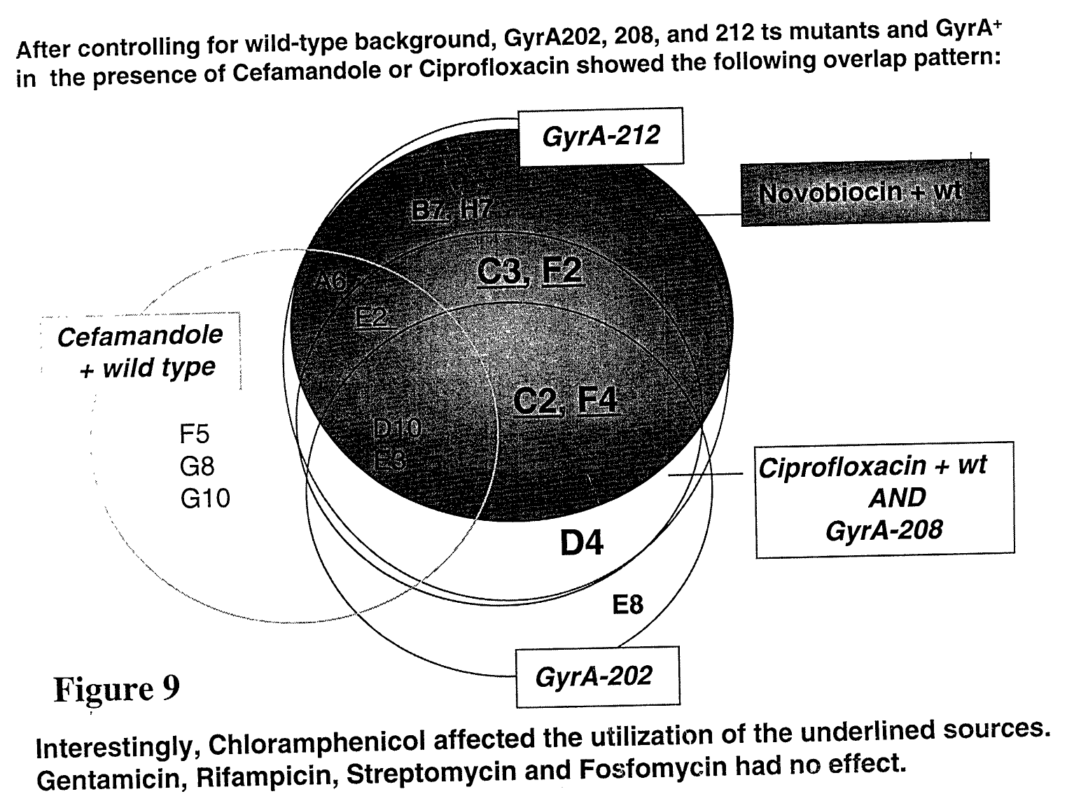

FIGS. 5-9 depict the utilization of individual carbon sources as indicators of specific inhibitors of bacterial growth. Antimicrobial agents with known mechanism of action are seen to display similar patterns of carbon utilization. This can be used to understand the mechanism of action of inhibitors with unknown mechanisms of action.

### Multi-channel Screening

The process of antimicrobial drug discovery consists either of modifications of existing antimicrobial agents (to enhance and improve on existing activities) or the process of identifying novel molecules (with unique activities). In this later process, the convention has been either to parse through collections of molecules or to de novo conceive of molecules which might have appropriate qualities. The former process has been substantially more rewarding. This screening process has been done using a wide variety of molecules from a variety of sources. Generally, screening libraries have come from organic chemical files, from natural product extracts and more recently from combinatorial chemical libraries. The object of the process is to include the most diverse set of molecules possible, searching for those molecules which have the biological activity of importance.

The screening process historically has been limited in the number of targets which could be examined at any time. Originally, antimicrobial screening was done in a “blind” fashion, exposing whole bacteria to the screening medium, looking for inhibition of growth as the positive report. Subsequently, screening came to the selection of a well founded target followed by extensive characterization of the target and the development of a biochemical screen designed to uncover inhibitors or potentiators of the target. This extensive characterization precluded the examination of more than a few targets at one time.

In most cases, a target was and is a microbial enzyme or process which if altered in its function by a small molecule or protein leads to dysfunction for the microorganism.

To accelerate the process for the discovery of novel antimicrobial agents we describe herein a method for the more effective discovery of antimicrobial agents through screening. This method employs a fundamentally genetic (as opposed to biochemical) technology which will accelerate the process and will allow the examination of multiple targets simultaneously. It also allows for the more effective characterization of possible effective agents, returning immediately information which will allow for the more effective prioritization of the collections of inhibitors.

In summary, the old, biochemical paradigm for antimicrobial agent screening was based on single assay systems and was designed to uncover molecules from the molecular diversity sources which would influence the activity of the biochemical target. This method returns a single activity report (on the target being examined) and requires substantial subsequent follow-up to validate and understand the importance of the inhibitor which was so discovered. We describe here a method to be used for the discovery of antimicrobial agents using multiple discriminators simultaneously. This method examines the activity of possible inhibitory molecules against multiple targets.

The multi-channel screening method provides more information on compounds of interest, more immediately. It identifies and culls out the most interesting molecules based on the pattern of biological activity for the compounds, and allows the discovery of better qualified activities. This method allows many targets to be examined and tested simultaneously. (See, FIG. 4 for examples of advantages.)

The method involves the use of a fundamentally genetic approach to antimicrobial drug discovery. Genetic mutants are used in the process. Mutants which are known to be important to the microorganism, such as temperature sensitive mutants (mutants which are not viable at a non-permissive temperature) are used to provide individual reports on the activity of possible novel antimicrobial molecules. The collection of the mutants, characterized by their requirement for function for in vitro or in vivo growth, collectively allows for the discrimination of possible inhibitor molecules by the differential growth effects of particular essential gene mutants (as compared to the wild-type parent strain). The pattern of response to any given possible inhibitor allows for the identification of the mechanism of action of the inhibitor, as well as the specificity of the possible inhibitors.

For example (see FIG. 1) three Salmonella typhimurium temperature sensitive mutants were used to show differential susceptibility specifically to the flouroquinolone DNA gyrase inhibitors. These mutants have been demonstrated to be mutants at the noted positions in the gyrA protein, the target of the quinolone antibiotics. The pattern of response was seen to be specific to the gyrase mutants, instructing on the mechanism of action (gyrase inhibition) of the quinolones. Thus, the method showed the utility of the temperature sensitive methodology, the power of the genetic screening method, and the ability to examine a much greater number of targets simultaneously (as compared to biochemical methods). The genetic potentiation in the case of the gyrase mutants was seen to be increased susceptibility to quinolones. This hypersensitivity provides the opportunity to uncover less potent inhibitors which could, through medicinal chemistry modifications, be effective antimicrobial agents. (See FIG. 1 for susceptibility profiles of gyrase mutants.)

This method can be extended to include the use of specific temperature sensitive mutants which act as indicators of inhibitions processes within defined biochemical pathways. In this example a temperature sensitive mutant whose mutation is in e.g. the cell wall biosynthesis biochemical pathway, can be used to identify compounds which are inhibitors of the specific enzyme in which the mutation is located (as per the gyrase example above) or inhibitors of steps in the biochemical pathways which precede or antecede the mutant enzyme. In this example inhibitors which are influencing processes before the biochemical step in which the mutation is found more profoundly influence the mutant cells growth (over the wild-type parent) (See, FIG. 2a, 2b).

Antibiotic potentiation also extends the dynamic range of multi-channel screening. Subinhibitory (to growth) amounts of antibiotics which have known mechanisms of action can be added to the temperature sensitive mutants (mutant in known or unknown essential genes). The potentiation of the activity of the known antibiotics, bringing about the inhibition of the mutant in the presence of the previously subinhibitiory concentration of the known antibiotic can be used to help discriminate between test hypothetical inhibitors.

The mutant need not be characterized, as it is in the gyrase example. Knowing the essential nature of the gene (through the temperature sensitive phenotype) is sufficient for inclusion in the screening array. Both known (through sequence identity or homology with other known essential genes) and previously unidentified essential genes can be included in the panel used for screening. The screening panel will consist of multiple such mutants. The mutants identified as homologous to known essential genes will be classified and grouped by function, e.g. DNA metabolism mutants, cell wall biosynthesis mutants, cell division mutants and so forth. Then putative inhibitory compounds will be exposed to these mutants. After growth of the mutants/parents alone and in combination with potentiation antibiotics at subinhibitory concentrations the inhibition of growth in the presence of the putative novel molecules will be examined. The format can be (but is not limited to) microtiter 96 well liquid cultures. The method was chosen for ease of manipulation and compatibility with conventional robotic automation. This method will allow for the examination of thousands of samples, against the variety of targets. (See, FIG. 3 for possible plate layout.)

### EXAMPLES

### Example 1

### MIC determination procedure: Micro Broth Dilution Technique

Referring to FIG. 1, the experiments were essentially by the methods outlined by the National Committee for Clinical Laboratory Standards (NCCLS). Bacteria were grown in Mueller-Hinton (MH) broth with agitation at 30° C.

The antibiotic dilution ranges used were:

Novobiocin: Range 128-0 μg/ml. Coumermycin A1: Range 128-0 μg/ml. Ciprofloxacin: Range 0.5-0 μg/ml. Norfloxacin: Range 8-0 μg/ml. Mitomycin C: Range 8-0 μg/ml Phenylmercuric acetate: Range 32-0 μg/ml. 4-Nitroquinoline Oxide: Range 32-0 μM Rifampicin: Range 128-0 μg/ml. Gentamicin: Range 32-0 μg/ml. Streptomycin: Range 128-0 μg/ml. Cefotaxime: Range 8-0 μg/ml. Ampicillin: Range 32-0 μg/ml. Phosphonomycin (or fosfomycin): Range 128-0 μg/ml.

The following list contains temperature sensitive Salmonella typhimurium mutants that were grouped based on their phenotype and/or map position of the mutation.

| | |
|---|---|
| Strain/Mutant: | Characteristics: |

| | | |
|---|---|---|
| 7389 | gyrA+ | wild type control |
| 7527 | gyrA202 | Gyrase subunit A |
| 7529 | gyrA208 | Gyrase subunit A |
| 7533 | gyrA212 | Gyrase subunit A |

The data in FIG. 1 shows that sensitivity of gyrA mutants to the antibiotics tested is specific and representative of the effect of the antibiotic on the microbial target.

### Example 2

### Differential Carbon Source Utilization

The ability to metabolize particular substrates, such as carbon or nitrogen sources, has been used to characterize microorganisms (Lederberg, 1948) for genetic screening (Gutnick et al., 1969; Alper and Ames, 1975) and indeed to classify microorganisms for the purpose of taxonomy (Bochner, 1992). Indicators of metabolism can be growth, pH, or redox-sensitive dyes. Below, we describe two applications in which the differential ability of a bacterial strain in the presence or absence of a test compound to utilize a particular substrate (or substrates) is exploited to create a screen that identifies new antimicrobial agents. In one application, a wild-type strain, when grown in the presence of the test compound, will recreate a phenotype of a mutant test strain that has been previously characterized and shown to differ from that of the wild-type strain (with respect to substrate metabolism). In another application, a wild-type strain, when grown in the presence of the test compound, will alter the profile of substrate utilization to recreate a previously characterized phenotype seen when the wild-type strain is exposed to a stressor (such as sub-MIC levels of antibiotic, sub-lethal levels of mutagens, etc.). Again, such a phenotype would have been previously characterized and shown to differ from that of wild-type.

Preliminary characterization of the utilization of large numbers of carbon sources can be carried out using manual or automated taxonomic screening devices, such as the Biolog MT, ES, GN or GP Microplate™. Alternatively, this can be performed using commercially available components. We have characterized Salmonella typhimurium wild type and temperature-sensitive mutants, or wild type with sublethal levels of stressor. Briefly, strains were swabbed onto TSA plates and grown at a permissive temperature of 30° C. for 6 hours to provide an inoculum. The inoculum (MacFarland standard ˜1 in 0.89% sterile saline solution) was prepared as per Biolog “Instructions for Use” handbook, with the exception that leucine was added to correct for mutants housed in strains which were in a leucine-deficient background. Inoculum was added to Biolog GN or ES plates and incubated at the semipermissive temperature of 35° C. overnight (˜21 hr). Carbon source utilization is read colorimetrically in a microplate reader at 600 nm. Substrates that are not used similarly by wild-type and mutant are identified. Preliminary identification of substrates that are differentially utilized by wild-type and mutant, or wild-type ± stressor, is followed by confirmation testing and optimization of the differential signal by varying concentration of substrate and other conditions (e.g., inoculum, temperature, media components). Typically, these steps were performed using Biolog MT plates or by using a redox indicator (such as tetrazolium violet, 0.0001%) in a minimal medium with a sole carbon source (substrate being tested), supplemented with a small amount of tryptone (0.06%) or proteose peptone (0.2%). It was possible to do this in 96-well microtiter plate format, which will be adaptable to high throughput or multichannel screening.

Carbon source utilization differed significantly from wild-type in a majority of mutants and stressor conditions tested. Two particularly illustrative cases are that of the GyrA mutants versus wild-type, and that of wild-type ± ciprofloxacin. First, as shown in FIGS. 5-9, three different gyraseA mutants differed from wild-type in their inability to use several carbon sources (the alphanumeric codes correspond to well locations on Biolog GN plates). This effect was significant in that a) the mutants resembled wild type in their ability or inability to use 70 out of 95 (70/95) carbon sources tested, differing only in their utilization of a combined total of 15/95 carbon sources tested, and b) there was very significant overlap in the phenotypes with respect to carbon source utilization of the three gyraseA mutants. Mutants in other genes produced significantly different phenotypes. Second, the inclusion of sub-MIC levels of ciprofloxacin, whose molecular target in the cell is known to be gyraseA, reproduces the phenotype of one of the mutant alleles perfectly, and has significant overlap (46-71%) with the other two. Other classes of antibiotics administered at sub-MIC levels produced different phenotypes from the gyraseA/ciprofloxacin induced phenotype. Exceptions were cefamandole and chloramphenicol (40% overlap). Interestingly, novobiocin, which targets gyraseB, produced a very similar phenotype to that of the gyraseA mutants and the ciprofloxacin induced phenotype.

While we described above two different applications, the two examples described herein bear a relationship to each other and allow further conclusions to be drawn. These examples reveal that a carbon utilization pattern, while not necessarily directly linked to a particular mutation or drug target, can provide a somewhat specific readout of the integrity of the function of that gene product or target. Thus, by screening as described, we can hope to identify agents whose mode of action in some way relates to the functional integrity of known gene products or known stressor targets.

References:

Alper, M. D., and B. N. Ames. 1975. Positive selection of mutants with deletions of the gal-chl refion of the Salmonella chromosome as a screening procedure for mutagens that cause deletions. J. Bacteriol. 121:259-266

Bochner, Barry. 1992. U.S. Pat. No. 5,134,063. Methods for detection, identification and specification of Listerias.

Gutnick, D., Calvo, J. M., Klopotowski, T., and B. N. Ames. 1969. Compounds which serve as the sole source of carbon or nitrogen for Salmonella typhimurium LT-2. J. Bacteriol. 100:215-219

Lederberg, J. 1948. Detection of fermentative variants with tetrazolium. J. Bacteriol. 56:695

### Example 3

### Examples of Possible Salmonella typhimurium Strain Groupings within a Multi-channel Screen Plate

1. For identification of putative test compounds acting on DNA metabolism:

Strain 7533 (gyrA212)

Strain 7818 (parF)

Strain 5174 (parF)

Strain 5178 (parF)

Strain 5041 (UV sensitive)

Strain 5066 (UV sensitive)

Strain 5051 (Filamentous cells)

(While specific strains are noted herein, equivalent strains are obtainable by those in the art; these strains are not limiting in the invention.) These strains will be mainly hypersensitive to test compounds acting on DNA metabolism but some will also be susceptible to test compounds acting on other cellular pathways. Consequently to increase even more the specificity, it is possible to include potentiation tests in the screening plate:

The effect of test compounds on this group of mutant can be confirmed by the inhibition of the wild-type strain grown in the presence of sublethal concentrations of toxic agents acting on DNA metabolism:

With the addition of the test compound, inhibition of

Wild type strain with sublethal concentration of mitomycin C, and/or,

Wild type strain with sublethal concentration of Phenylmercuric acetate, and/or,

Wild type strain with sublethal concentration of coumermycin Al, and/or,

Wild type strain with sublethal concentration of novobiocin will confirm the effect of the test on parF mutants and indicate a possible topoisomerase IV inhibitor;

inhibition of

Wild type strain with sublethal concentration of ciprofloxacin, and/or,

Wild type strain with sublethal concentration of norfloxacin

will confirm the effect of the test compound on gyrA212 and indicate a possible gyrase inhibitor;

inhibition of

Wild type strain with sublethal concentration of 4-Nitroquinoline oxide, and/or,

Wild type strain with sublethal concentration of rifampicin, and/or,

Wild type strain with suprainhibitory concentration of fosfomycin

will confirm the effect of the test compound on UV sensitive mutants and filamentous mutant 5051 and indicate a possible inhibitor or DNA repair and maintenance.

Also, the effect of test compounds on the gyrA212 mutant can also be confirmed by the lack of metabolism of formic acid by the wild type strain in the presence of the test compound. Indeed, the utilization of this carbon source marker was shown to be affected by sub-MICs of DNA gyrase inhibitors such as ciprofloxacin.

2. For identification of putative test compounds acting on cell wall metabolism:

Strain 7587 (dapA)

Strain 5119 (murCEFG cluster)

Strain 5091 (Thymidine incorporation defect)

These strains will be hypersensitive to test compounds acting on cell wall metabolism but some will also be susceptible to test compounds acting on other cellular pathways. Consequently to increase even more the specificity, it is possible to include potentiation tests in the screening plate:

The effect of test compounds on this group of mutant can be confirmed by the inhibition of the wild-type strain grown in the presence of sublethal concentrations of toxic agents acting on cell wall synthesis:

With the addition of the test compound, inhibition of

Wild type strain with sublethal concentration of ampicillin, and/or,

Wild type strain with sublethal concentration of cefotaxime will confirm the effect of the test compound on these mutants and indicate a possible inhibitor of cell wall metabolism;

The effect of test compounds on this group of mutants can also be confirmed by the lack of metabolism of D-alanine or D-serine by the wild type strain in the presence of the test compound. Indeed, the utilization of these two carbon source markers was shown to be affected by sub-MICs of cell wall synthesis inhibitors such as cefamandole.

3. For identification of putative test compounds acting on protein metabolism:

Strain 5258 (parE)

Strain 8041 (parF)

Strain 5174 (parF)

Strain 5178 (parF)

These strains will be hypersensitive to test compounds acting on protein metabolism but some will also be susceptible to test compounds acting on other cellular pathways. Consequently to increase even more the specificity, it is possible to include potentiation tests in the screening plate:

The effect of test compounds on this group of mutant can be confirmed by the inhibition of the wild-type strain grown in the presence of sublethal concentrations of toxic agents acting on protein metabolism:

With the addition of the test compound, inhibition of

Wild type strain with sublethal concentration of gentamcin, and/or,

Wild type strain with sublethal concentration of streptomycin, and/or,

Wild type strain with sublethal concentration of phenol will confirm the effect of the test on these mutants and indicate a possible inhibitor of protein metabolism;

4. For identification of putative test compounds acting on yet unidentified essential cellular targets:

Strain 7585 (Odd shape phenotype)

Strain 5208 (filamentous cells)

Strain 7141 (filamentous cells)

Strain 5052 (filamentous cells)

These strains were not hypersensitive to known antibiotic or toxic agents although they have a yet unknown crippled essential cell function. They can be used in the screen to identify test compound acting on on yet unidentified essential cellular targets by showing hypersusceptibility to the test compound.

Uses

As is evident from the description above the methods of this invention are useful for rapid screening of biomolecule modulators. In addition, they are useful for determining the mechanism of action of any such modulator on a microbe. Such information is useful in general chemical screening for new antimicrobial agents and for routine laboratory testing of microbial sensitivity to such agents. In addition, the methods are useful for aiding rapid analysis of the mechanism of action of any particular agent which is useful for obtaining approval of such agents in therapeutic protocols.

Other embodiments are within the following claims:

1. Method for screening for a modulator of a biomolecule comprising the step of comparing growth of a first microbe having an altered said biomolecule and a second microbe having a normal said biomolecule wherein said first and second microbes are in contact with a potential modulator in a growth medium.

2. The method of claim 1 wherein said comparing is performed in a plurality of different media differing in their carbon source.

3. The method of claim 1, wherein a plurality of said first microbes having different altered said biomolecules are compared to said screened microbe.

4. Method for determining a fingerprint of a microbe having a biomolecule comprising the steps of comparing the growth of one or more microbes having an altered said biomolecule in a plurality of different media differing in their carbon source.

5. The method of claim 1 or 4 wherein said biomolecule is an enzyme.

6. The method of claim 1 or 4 wherein said alteration is a defect in said biomolecule which lowers the activity of said biomolecule.

7. The method of claim 6 wherein said defect is a temperature sensitive defect.

8. The method of claim 1 or 4 wherein said growth is measured by an indirect method.

9. The method of claim 1 or 4 wherein said microbe is selected from the group consisting of the Staphylococci, the Pseudomonads, the Enterococci, and the Streptococci.

10. The method of claim 1 or 4 wherein said altered biomolecule is overexpressed.

11. The method of claim 1 or 4 wherein said altered biomolecule is expressed at a lower level than the wold-type analog of said biomolecule.
